(19) United States
(12) Patent Application Publication
(10) Pub. No.: US 2025/0076067 A1
(43) Pub. Date: Mar. 6, 2025

(54) COMPUTER IMPLEMENTED METHOD AND DEVICE FOR ASSISTING PERSON TO PERFORM A TASK AND COMPUTER PROGRAM PRODUCT

(71) Applicant: Honda Motor Co., Ltd., Tokyo (JP)

(72) Inventors: Matti Krüger, Offenbach (DE); Jens Schmüdderich, Offenbach (DE)

(73) Assignee: Honda Motor Co., Ltd., Tokyo (JP)

(21) Appl. No.: 18/458,125

(22) Filed: Aug. 29, 2023

(51) Int. Cl. G01C 21/36; A61B 5/024; H04S 7/00

(52) U.S. Cl. CPC G01C 21/3629; A61B 5/024; H04S 7/304; H04S 2400/11; H04S 2420/03

(57) ABSTRACT

The disclosure relates to a method for assisting person to perform a task. The method includes: acquiring location information or state information on a location of the assisted person, and acquiring target location information on a target location based on a spatial target of the assisted person or target state information on a target state of the assisted person. The method generates a spatial signal based on the acquired location information and target location information, or based on the acquired state information and target state information. The method proceeds with obtaining an audio output signal for reproduction, and modulating the obtained audio output signal with the spatial signal. A perceived position of origin or a direction of the modulated audio output signal corresponds to the position information or the direction information included in the spatial signal. Then the modulated audio output signal is output to the assisted person.

### BACKGROUND

### Technical Field

The disclosure relates to the general field of assistance systems for assisting persons, in particular in mobility-related tasks, for example to assistance systems for operating vehicles or personal navigation systems for pedestrians or cyclists. The disclosure also relates to assistance systems for personal training applications, fitness applications, health monitoring, and biofeedback.

### Related Art

Assistance systems for mobility-related tasks, such as assisting an operator in traffic navigation exist in the automotive, aeronautical or maritime domain. Assistance systems can contribute positively to an operator's situation assessment, to his navigation skills and therefore to safety in the mobility environment, in which the assisted person operates.

An environment that contains multiple elements and is dynamically changing requires a high degree of attention of a person operating in that environment. For example, in case the person is controlling a vehicle (“ego-vehicle”) while simultaneously listening to music reproduced by an infotainment system of the ego-vehicle, the music may interfere with sound from the environment that includes relevant information for performing the driving task. Perceiving the sound from the environment with the relevant information is therefore more difficult for a person, whose attention will be occupied already with coping with visual information from the surrounding environment.

In the dynamic environment such as a current road traffic environment, complex situations with multiple choices for a next action to be performed by the person driving the ego-vehicle occur. The person may have difficulties in selecting one particular action that maximizes utility from the plurality of possible actions if not all relevant information available has been considered during the selection process. When, for example, a vehicle immediately preceding the ego-vehicle on a road with plural lanes in one driving direction decelerates, the person driving the ego-vehicle may have to choose between slowing down the ego-vehicle, possibly at the expense of energy efficiency and changing to another available lane. The choice depends on information on a current as well as a predicted occupancy of neighboring lanes in near future. However, a currently existing gap enabling a change of the ego-vehicle onto a neighboring lane may no longer be available at decision time, if the person due to the dynamically changing traffic scenario in the dynamic environment does not acquire information on the currently available gap fast enough.

Concerning the road traffic environment, there exist currently approaches to enhance existing vehicles by addition of wearable devices. Using wearable devices may even increase safety in vehicles.

Patent application publication US 2017/0156000 A1 discloses a wearable earpiece configured to connect wirelessly with an ego-vehicle. Having connected with the vehicle, the earpiece enters a driving mode. In the driving mode, the earpiece senses ambient sound with a microphone associated with the earpiece and reproduces the sensed ambient sound with its speaker. The earpiece may combine audio streams comprising ambient sound from within the vehicle cabin and from outside the vehicle cabin with further audio streams from entertainment or infotainment systems and audio messages associated with vehicle state information or vehicle alert conditions. An intelligent control system may modify audio characteristics of the reproduced audio streams, in particular reduce or amplify an amplitude of the ambient sound or portions thereof. Modifying audio characteristics of the reproduced audio streams may depend on detecting particular actions of the driver of the vehicle, or for providing audio cues regarding presence or absence of other vehicles in the environment of the ego-vehicle.

Alternatively, EP 4 163 896 A1 discloses integrating a human-machine interface into an assistance system that utilizes spatial sound modulation to change a perceived location of the origin of speaker or earphone-produced sounds in an assistance system in order to convey predicted risks in a dynamic and rapidly changing traffic environment. However, EP 4 163 896 A1 is restricted to conveying information on the risks identified by the assistance system. There remain issues of assisting a person in performing a task in a dynamically changing environment that supports the assisted person in focusing the attention towards scene elements relevant to the task and independent on their risk potential with regard to the task, simultaneously the assistance yielding a high acceptance of the assistance from the perspective of the assisted person.

### SUMMARY

The present disclosure proposes a computer-implemented method for assisting a person according to claim 1, a computer program product comprising machine-readable instructions and an assistance device according to the corresponding independent claims.

The computer-implemented method for assisting a person to perform a task comprises a step of acquiring location information on a location of the assisted person or state information on a state of the assisted person. The method further comprises a step of acquiring target location information on a target location based on a spatial target of the assisted person or target state information on a target state of the assisted person. The method generates a spatial signal based on the acquired location information and the acquired target location information, or based on the acquired state information and the acquired target state information. The spatial signal includes a position information or the direction information. The method proceeds with obtaining an audio output signal for reproduction to the assisted person, and modulating the obtained audio output signal with the spatial signal, wherein a perceived position of origin or a direction of the modulated audio output signal corresponds to the position information or the direction information included in the spatial signal. Then the modulated audio output signal is output to the assisted person. Preferably, the audio output signal is signal output by an infotainment system, but also covers, for example, an output signal for a conversation on the phone utilizing a headset.

The dependent claims define further advantageous embodiments.

### BRIEF DESCRIPTION OF THE DRAWINGS

The aspects and implementation of the present disclosure will be explained in the following description of specific embodiments in relation to the enclosed drawings, in which:

FIG. 1 displays a simplified flowchart illustrating an overview over the method for assisting a person according to a first embodiment;

FIG. 2 displays a simplified flowchart illustrating an overview over the method for assisting a person according to second embodiment;

FIG. 3 displays a block diagram of an assistance system implementing an embodiment;

FIG. 4 illustrates an embodiment with an encoding adapted to limited hardware;

FIG. 5 presents a first exemplary scenario of applying the assistance method in a traffic environment;

FIG. 6 presents a second exemplary scenario of applying the assistance method in a traffic environment;

FIGS. 7A to 71 present exemplary scenarios of applying the assistance method in a public traffic environment.

The description of figures uses same references numerals for same or corresponding elements in different figures. The description of figures dispenses with a detailed discussion of same reference numerals in different figures whenever considered possible without adversely affecting comprehensibility.

### DETAILED DESCRIPTION

The method for assisting a person according to the first aspect is advantageous as the method extends the known HMI to a utilization of spatial sound modulation to change a perceived location of speaker or earphone-produced audio output to a perceived location that is set to correspond to a target location, a target direction or a target state that is determined by the application context. In the application of navigation assistance, the perceived location may correspond to the current location of the assisted person, as long as the assisted person is on the target path, the intended path or recommended path, or at least within an intended region (section) on the target path. The perceived location may correspond to a predicted future location of the assisted person on the target path, the intended path or recommended path, or within an intended region (section) on the target path.

For assistance in navigation, there may exist embodiments, which benefit from the output sound appearing to originate from a perceived location slightly moving ahead, e.g., in intersections scenarios, in order to provide information about where to go, when arriving at the intersection. Such cases are included in the term target state or target location” to include a location where the ego-vehicle is predicted or estimated to be within a predetermined time, e.g., some seconds, from the current time. In case a predicted vehicle trajectory is aligned with the ego-vehicle reaching the target location or target state in the predetermined time of, e.g., some seconds, the sound does not change. In case of intersections ahead on the predicted trajectory of the ego-vehicle, there lacks sometimes certainty about where the assisted person acting as driver of the ego-vehicle will go, unless e.g., indicated by indicator lights early. In that case, the output sound could move the perceived point of origin ahead on the predicted trajectory in order to indicate a desired future location or direction to the assisted person.

The method overcomes issues with conventional navigation system interfaces relying on visual display of navigation information that concern the blocking of visual senses by task relevant actions in the dynamic environment, e.g., operating an ego-vehicle. Speech output of navigation information represents an alternative approach. However, the presented assistance method employs spatial sound changes as a particular fast and intuitive alternative that is both fast as well as subtle, making the assistance method advantageous for many application scenarios.

The assistance method is applicable in a wide range of scenarios that include vehicle-bound applications that utilize, for example, infotainment systems with two or more loudspeakers for the interior of the vehicle. Alternatively, the method may be applied with headrest speakers, helmet mounted loudspeakers or generally similar headphone arrangements in case of motorcycles or bicycles. In a further alternative, the assistance method may be applied using an audio system with a mono sound output only. The infotainment system may also be part of a portable music player or smartphone.

Generally, the method utilizes a mobile audio output device, e.g., a binaural loudspeaker assembly capable to provide sound to the assisted person, wherein the assisted person is able to perceive a direction of origin or a position of origin of the reproduced sound. The method uses localization information of the assisted person, as available in current navigation systems or smart devices. The method makes an advantageous use of sound that is not directly related to perform the primary task. The primary task corresponds, but is not limited to moving from a start location or a current location to a predetermined destination. In the case of pedestrians, available information sources may be location services including satellite based navigation systems, e.g., GPS, already available, which enable to determine any actual deviation from a predetermined path. In the future, shared information from vehicles and the environment may be used as information sources. A “predetermined destination” may even be hypothesised destination, which is a destination inferred, for example, from observation of past behavior. For example, for a commuter who tends to use the same route on most days preferably at the same time of the day, a system might assume this specific destination without the user having to set them explicitly. Thus, from observations on destinations used in the past, the system may conclude that one of these destinations shall be addressed again. The selection of such known destination from the past may take into consideration, for example, a comparison of the current position and time with previous starting positions and times stored in association with each particular destination.

The method enables to modify a perceived spatial location of the audio information output as directed output sound to the assisted person in order to support the assisted person in range of aspects: e.g., the directed output sound enables the assisted person to stay on a predetermined route. The assisted person may also be guided to return to the predetermined route. The method may also support the assisted person in selecting a preferred direction, e.g., at an intersection. The method also enables support for the assisted person in identifying alternative routes to the destination. Moreover, the method may also assist in identifying navigation destinations or specific points of interest (POI). Using spatial sound modulation allows conveying the information to the assisted person in a subtle and intuitive manner without interfering with visual perception. Thus, conveying information using directivity of the audio output sound neither burdens nor blocks the visual perception of the assisted person, which is particularly advantageous while operating any kind of vehicle.

Applying the disclosed assistance method in a mono setup may be advantageous in scenarios of one-dimensional mobility, e.g., including but not limited to trains, or simply the assisted person walking or driving along a path. The perceived location of origin can then be set towards the front of the assisted person in the direction of travel. The modulation of the output sound includes varying distances of the perceived location of origin in order to guide the assisted person in adapting his or her velocity.

According to an embodiment of the method, the step of generating the spatial signal, the spatial signal is generated based on a deviation of the acquired location information from the acquired target location information, or based on a deviation of the acquired state information and the acquired target state information.

A deviation of the of the acquired location information from the acquired target location information indicates a deviation of the assisted person from a recommended path and results in spatial sound displacement, which the assisted person perceives.

The spatial signal may be generated based on a deviation of the predicted location information from the corresponding acquired target location information, or based on a deviation of the acquired predicted state information and the corresponding acquired target state information.

Basing generation of the spatial signal on an estimated (near) future location deviating from a (near) future target location is advantageous, as for many mobility-related tasks information about the near future is particularly advantageous and even more helpful than information about the current state. A human moving, and in particular when driving a vehicle at speed, usually checks not his current position but an estimated future position where he or she will be after a certain time, e.g., two seconds, has elapsed. This is in particular advantageous, as the assisted person may still influence with respective actions a future evolvement of the current scenario, while the current scenario is already determined.

The position information may, in an embodiment of the method, correspond to the current location of the assisted person, in case of determining that the deviation of the acquired location information from the acquired target location information, or the deviation of the acquired state information and the acquired target state information is below a first predetermined threshold or zero.

In an embodiment, generating the spatial signal includes setting the position information to correspond to a location ahead of the location of the assisted person in a moving direction of the assisted person.

Generating the spatial signal may include, in a further embodiment, setting the position information to correspond to a location in a direction of the target location from the location of the assisted person in case of determining that a moving velocity of the assisted person decreases or that the moving velocity of the assisted person is below a second predetermined threshold or the assisted person stops.

The state of the assisted person may include an ego-velocity of the assisted person, and the target state may include a predetermined velocity target.

Determining a deviation of an ego-velocity (velocity of the assisted person) from the target velocity enables to modulate the perceived audio output information in order to induce the assisted person to maintain the target velocity at a specific target velocity or pace. Maintaining a target velocity represents a goal that is common for physical training applications, e.g., running at a target speed that aligns with a personal training goal. Many people listen to sound including music while performing mobility exercises, such as running. Spatial sound changes applied to the reproduced music may provide the person with an additional motivation to reach their target velocity, without interrupting their listening and exercise experience, as they may prefer music to appear spatially centered.

The state of the assisted person may include a safety-related variable associated with the assisted person, and the target state may include a predetermined target value of the safety-related variable.

The state of the assisted person may include a fitness-related variable or a health-related variable associated with the assisted person, and the target state may include a predetermined target value of the fitness-related variable.

The computer-implemented method, according to an embodiment, has the fitness-related variable or the health-related variable including a heart rate of the assisted person, and the predetermined target value of the fitness-related variable or health-related variable including a predetermined heart rate target. The method further comprises generating the spatial signal based on the heart rate and the acquired target heart rate, wherein the spatial signal includes setting the position information or the direction information based on the deviation of the heart rate and the acquired target heart rate.

According to an embodiment, the computer-implemented method may include generating the spatial signal based on the heart rate and the acquired target heart rate, determining the position information or the direction information based on the deviation of the heart rate and the acquired target heart rate, and further based on a pace target value.

According to an embodiment of the computer-implemented method, the assisted person moves in a dynamic traffic environment including a plurality of other participants, the safety-related variable includes a distance of the assisted person to the other traffic participants and the predetermined target value of the safety-related variable includes a minimum distance of the assisted person to the other traffic participants.

The computer-implemented method may have the assisted person wearing a virtual reality headset for moving in a virtual representation and reproducing the audio output signal associated with the virtual representation. The safety-related variable includes a location of the assisted person in a real environment of the assisted person, and the predetermined target value of the safety-related variable includes a safe location of the assisted person.

The method thereby supports the assisted person acting in the virtual reality environment in staying within a safe space of the real environment while using the virtual reality headset. In this application, utilizing a spatial shifting of ambient sounds within the virtual environment guides the assisted person back to a desirable region or safe region in the real environment. The subtle approach of changing the perceived location of origin of the audio output may even achieve this beneficial effect without breaking the immersion of virtual reality.

According to an embodiment, the computer-implemented method includes outputting the modulated output signal via plural speakers or via earphones worn by the assisted person.

The computer-implemented method according to according to an embodiment comprises a step of unified encoding of multiple objectives in the target state information. Individual contributions of the objectives are weighted differentially based on at least one of priority constraints or preset user preferences.

Hence, the computer-implemented method provides a technical implementation for managing different, possibly even contradicting objectives and targets for the assisted person in a technical implementation of a computer implemented method or an assistance device, which provide on the other hand an intuitive assistance to the person using the modulated output signal.

According to an embodiment, the computer-implemented method includes the target state information encoding objectives that include at least one of safety objectives, navigation objectives, sports objectives, health objectives, and convenience objectives.

Thus, the modulated output signal may assist the person in a wide range of advantageous applications, which combine and prioritize different targets set by the assisted person.

In the computer-implemented method according to according to an embodiment, the priority constraints include a legally required preference for safety objectives over convenience objectives.

Hence, the method provides the user with an assistance that integrates multiple and contradicting objectives by simultaneously being based on the assistance output in the modulated output signal automatically on priorities that guard the health of the assisted person. The quality of the assistance is significantly improved with regard to existing assistance systems.

An embodiment of the computer-implemented method comprises steps of determining a reaction or a lack of reaction of the assisted person to a previously output modulated audio output signal modulated based on a same spatial signal; and adapting a parameter of the modulated audio output signal based on the determined reaction or lack of reaction of the assisted person.

Thus, the computer-implemented method is capable to assist the person with a high degree of situation-awareness and in a manner adapting to a specific evolvement of a situation in the environment and a capability of the assisted person to take account of the evolvement of the situation.

An embodiment of the computer-implemented method comprises in the step of acquiring location information, acquiring orientation information of an orientation of the assisted person in relation to at least one of the location of the assisted person and a target direction of the assisted person. The embodiment further comprises, in the step of generating the spatial direction signal, generating the spatial direction signal further based on the acquired orientation information.

Acquiring orientation information of the assisted person in relation to a target location of the assisted person, or a direction from the location of the assisted person to the target location has the effect of enabling to determine a spatial shift included in the spatial direction signal in a correct relation to the assisted person's orientation. If, for example, the assisted person wears headphones or earphones for outputting the modulated audio output signal to the assisted person, the orientation information may include orientation information of the spatial orientation of the head of the assisted person.

In a second aspect of the disclosure, a computer program product comprising machine-readable instructions executable by a digital processor to cause the digital processor to control a method for assisting a person to perform a task, the method comprises a step of acquiring location information on a location of the assisted person or state information on a state of the assisted person. The method further comprises a step of acquiring target location information on a target location based on a spatial target of the assisted person or target state information on a target state of the assisted person. The method generates a spatial signal based on the acquired location information and the acquired target location information, or based on the acquired state information and the acquired target state information. The spatial signal includes a position information or a direction information. The method proceeds with obtaining an audio output signal for reproduction to the assisted person, and modulating the obtained audio output signal with the spatial signal, wherein a perceived position of origin or a direction of the modulated audio output signal corresponds to the position information or the direction information included in the spatial signal. Then the modulated audio output signal is output to the assisted person.

A device for assisting a person in performing a task according to a third aspect of the disclosure comprises at least one sensor configured to acquire location information on a location of the assisted person or state information on a state of the assisted person, and at least one interface configured to acquire target location information on a target location based on a spatial target of the assisted person or target state information on a target state of the assisted person. The device further comprises at least one processor configured to generate a spatial signal based on the acquired location information and the acquired target location information, or based on the acquired state information and the acquired target state information, wherein the spatial signal includes a position information or a direction information. The processor is further configured to obtain an audio output signal for reproduction to the assisted person, to modulate the obtained audio output signal with the spatial signal. A perceived position of origin or a direction of the modulated audio output signal corresponds to the position information or the direction information included in the spatial signal. The processor is yet further configured to control outputting the modulated audio output signal to the assisted person via plural speakers or via earphones worn by the assisted person.

The computer program according to the second aspect and the assistance system according to the third aspect achieve same or corresponding advantages as discussed above with regard to the method according to the first aspect.

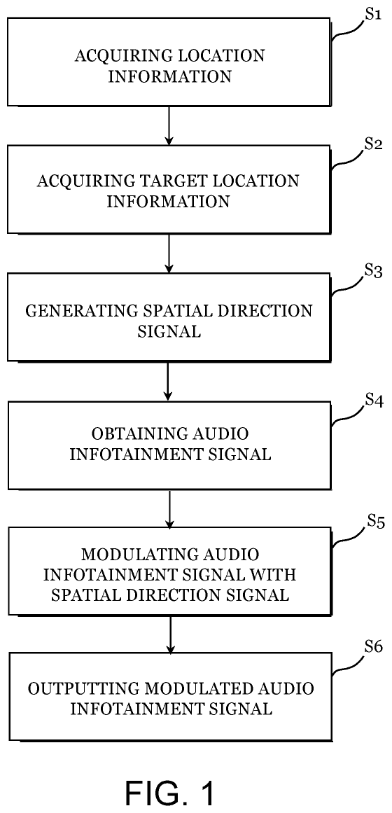

FIG. 1 displays a simplified flowchart illustrating an overview of the method for assisting a person according to a first embodiment.

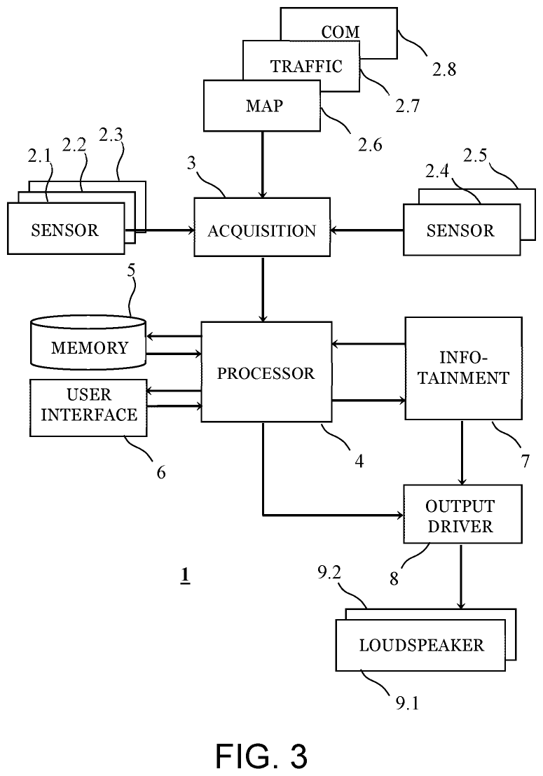

The method is a computer-implemented method for assisting a person to perform a task. The task may include travelling from a starting point (start location) to a destination via a predetermined route. The route may be computed and proposed by a navigation system based on the start location and the destination input by the assisted person, further using predetermined map data. Computing the proposed route may further be based on auxiliary information relevant to navigation including, e.g., at least one of past and current traffic data. A computer, for example an onboard electronic control unit (ECU) of a vehicle (ego-vehicle 10) or a processor 4 of a mobile personal communication device, e.g., a smartphone, may perform the steps of the method utilizing the associated input/output devices as discussed with reference to FIG. 3 below.

The method comprises a step S1 of acquiring location information that includes location data on a location of the assisted person. The location data may in particular define a current location of the assisted person. The method may acquire the location data via a localization sensor, e.g., a global navigation satellite system (GNSS) receiver such as a GPS receiver or a Galileo receiver.

The method further acquires in step S2 target location information on a target location based on a spatial target of the assisted person. The target location information may include target location data that is location information on a target position at which the assisted person intends to arrive. The assisted person may input the target position via a user interface 6. Alternatively, the target position may be acquired or estimated automatically, e.g., based on past driving logs or on calendar information. The processor may obtain the target information from a navigation system that generates route information based on map data, a start location and the target location. The target information may include location information associated with a time, e.g., a current time.

In step S3 following to steps S1 and S2, the method proceeds with generating a spatial signal based on the acquired location information and the acquired target location information. The generated spatial signal includes a position information or a direction information. In particular, the spatial signal may include information on a deviation of the acquired location information from the acquired target location information.

In step S4, the method obtains an audio output signal for reproduction to the assisted person. The audio output signal may include an audio stream, which the assisted person selected for reproduction via loudspeakers 9.1, 9.2 by an infotainment system 7 in the ego-vehicle 1, or via headphones by a smartphone or another type of mobile computing device, e.g., a tablet computer or a smart wearable device, e.g., a smart watch.

In step S5, the method modulates the obtained audio output signal with the spatial signal. By modulating the obtained audio output signal with the spatial signal in order to generate the modulated audio output signal, a perceived position of origin or a direction of the reproduced modulated audio output signal corresponds to the position information or the direction information included in the spatial signal. The modulated audio output signal is subsequently provided to respective acoustic output devices, e.g., the loudspeakers 9.1, 9.2 in the ego-vehicle 1, or the headphones for reproduction.

In step S6 following to step S5, the modulated audio output signal is output, e.g., via the output devices, to the assisted person. The reproduced modulated audio output signal has the perceived position of origin or a direction of the reproduced modulated audio output signal that corresponds to the position information or the direction information included in the spatial signal.

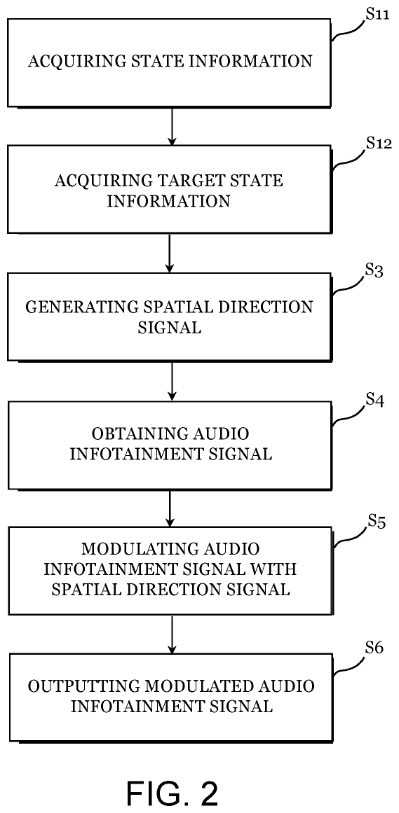

FIG. 2 displays a simplified flowchart illustrating an overview of the method for assisting a person according to a second embodiment. Like the first embodiment, the second embodiment includes a computer-implemented method for assisting a person to perform a task. The task may include travelling from a starting point (start location) to a destination via the proposed route and using a predefined means of transportation or even by walking. The proposed route may be computed by a navigation system based on the start location and the destination input by the assisted person, further using predetermined map data. The computer, for example the onboard electronic control unit (ECU) of a vehicle (ego-vehicle 10) or the processor 4 of a mobile personal communication device, may perform the steps of the method utilizing the associated input/output devices as discussed with reference to FIG. 3 below. The second embodiment is generally similar to the first embodiment; in particular, the processing sequence of steps S3 to S6 essentially corresponds to the steps S3 to S6 discussed with reference to FIG. 1.

The method comprises a step S11 of acquiring state information on a state of the assisted person. The state information may in particular define a current state of the assisted person. The method may acquire the state information via a localization sensor, e.g., a GNSS receiver or a vehicle sensor such as a velocity sensor. The method may further acquire state information via an orientation sensor, e.g., a gyroscope, in particular for embodiments in which heading information is required.

The method further acquires in step S12 target state information on a target state of the assisted person. The target state information may include target state data that is state information on a target state, which currently should apply for the assisted person. The assisted person may input the target state information via a user interface 6, or the target state information may be generated based on the user input. The processor may obtain the target state information from a driving assistance system that generates the target state information based on traffic data or based on a calculated proposed route that includes velocity information for each segment of the proposed route. The target information may include state information associated with a time, in particular a current time.

In step S3 following to steps S11 and S12, the method proceeds with generating a spatial signal based on the acquired state information and the acquired target state information. The generated spatial signal includes a position information or a direction information. In particular, the spatial signal may include information on a deviation of the acquired state information from the acquired target state information. In a specific example, for generating the spatial signal in step S3, the method may determine a deviation of the acquired state information of the assisted person from the target state information for the assisted person, e.g., by determining a difference between the acquired state information of the assisted person of step S11 from the acquired target state information for the assisted person of state S12. More specifically, in case of the state information including an ego-velocity of the assisted person and the target state being a target velocity, the method may determine a velocity difference between the ego-velocity and the target velocity, and whether the ego-velocity is smaller than the target velocity, equal to the target velocity or exceeds the target velocity.

For generating the spatial signal in step S3, the method may determine whether there is a deviation of the acquired state information of the assisted person from the target state information for the assisted person that exceeds a predetermined threshold. If the determined deviation is below the predetermined threshold, the spatial signal may set the position information or the direction information of the spatial signal to default values, e.g., the spatial signal does not modulate the audio output signal. Not modulating the audio output signal means here that the perceived position of origin or perceived direction of the reproduced the audio output signal remains unchanged for the modulated audio output signal when compared with the obtained audio output signal (original audio output signal).

If the determined deviation exceeds the predetermined threshold, the spatial signal may set the position information or the direction information of the spatial signal to corresponding position values or direction values, e.g., the spatial signal does modulate the audio output signal.

Setting the corresponding position values or direction values may include a processor 4 reading the corresponding position values or direction values from a stored correspondence table using the determined deviation as input or computing the corresponding position values or direction values using a predetermined function using the determined deviation as input to the predetermined function.

In step S4, which corresponds to step S4 of the first embodiment, the method obtains an audio output signal for reproduction to the assisted person. The audio output signal may include an audio stream, which the assisted person selected for reproduction via loudspeakers 9.1, 9.2 by an infotainment system 7 in the ego-vehicle 1, or via headphones by a smartphone or another mobile computing device.

In step S5, which corresponds to step S5 of the first embodiment, the method modulates the obtained audio output signal with the spatial signal. By modulating the obtained audio output signal with the spatial signal in order to generate the modulated audio output signal, a perceived position of origin or a direction of the reproduced modulated audio output signal corresponds to the position information or the direction information included in the spatial signal. The modulated audio output signal is subsequently provided to respective acoustic output devices, e.g., the loudspeakers 9.1, 9.2 in the ego-vehicle 1, or the headphones for reproduction.

In step S6, which corresponds to step S6 of the first embodiment, and which follows to step S5, the modulated audio output signal is output, e.g., via the output devices, to the assisted person. The reproduced modulated audio output signal has the perceived position of origin or a direction of the reproduced modulated audio output signal that corresponds to the position information or the direction information included in the spatial signal.

FIG. 3 displays a block diagram of an assistance system implementing an embodiment.

The block diagram provides an overview of structural elements of an assistance system 1 according to an embodiment. In the context of FIG. 3, the assisted person is a driver of the ego-vehicle 2.

In particular, FIG. 3 displays major units (modules, means) of the assistance system 1 that modulates the salience of sound output by an infotainment system 7 into the interior of the ego-vehicle 10, which is equipped with the assistance system 1. The sound output by the infotainment system 7 comprises audio output information (infotainment sound), e.g., radio audio streams, music streams, and/or audio streams provided by a vehicle navigation system not shown in FIG. 2. The sound output by the infotainment system 7 further comprises multimodal stimuli relating to traffic elements in the environment exterior of the ego-vehicle 10.

The assistance system 1 comprises an acquisition unit 3. The assistance system 1 acquires via the acquisition unit 3 information on the environment of the ego-vehicle 10, which is a dynamic environment characterized by changing traffic scenarios the ego-vehicle 10 and its driver corresponding to the assisted person have to navigate.

The acquired information can include sensor data acquired by at least one sensor 2 (sensor device) mounted on the ego-vehicle 10 and which senses the environment of the ego-vehicle 10. The sensors 2 may comprise active and/or passive sensors, e.g., camera sensors 2.1, radar sensors 2.2, lidar sensor 2.2, acoustic sensors, ultrasonic sensors, temperature sensors in any combination.

The acquisition unit 3 may further acquire the data including sensor data acquired by at least one sensor 2 mounted on the ego-vehicle 10 and which senses the interior of the ego-vehicle 1. Sensors acquiring sensor data from the interior of the ego-vehicle 10 may comprise active and passive sensors, e.g., camera sensors 2.4, acoustic sensors 2.5 (microphone), or temperature sensors in any combination.

The acquisition unit 3 may further acquire data including map data from a map data provider 2.6.

Additionally or alternatively, the acquisition unit 3 acquires data including traffic data from a traffic data provider 21.7.

Additionally or alternatively, the acquisition unit 3 acquires data including x-to-vehicle data via a vehicle communication module 2.8. The vehicle communication module 2.8 may, for example, enable the driver assistance system 1 to acquire data from other vehicles 11 in the environment of the ego-vehicle 10 via vehicle-to-vehicle communication, and also from traffic infrastructure devices capable of infrastructure-to-vehicle communication. The vehicle communication module 2.8 can include, for example, at least one wireless communication module operating according to a specific cellular communication standard such as LTE, LTE-Advanced, and 5G.

Additionally or alternatively, the acquisition unit 3 acquires sound data (audio output information) including at least one sound data stream from an infotainment system 3.

The assistance system 1 of FIG. 3 comprises a processor 4 (electronic control unit, electronic data processing unit). The processor 4 may comprise a plurality of processing units arranged on the ego-vehicle 10, or arranged spatially separated from the ego-vehicle 10 and communicating with the ego-vehicle 10 via the communication unit 2.8, for example, and implement a distributed processing architecture.

The processor 4 stores and reads data from a memory 5. The memory 5 in FIG. 3 represents data storage capabilities for recording application data, for example, audio data streams in the form of audio files, or program code of the assistance system 1, or data generated and stored during execution of the program code. The memory 5 thus also performs as a RAM or ROM memory.

The assistance system 1 may additionally include a user interface 6. The user interface 6 enables the assisted person to provide input data to the assistance system land to receive data output from the assistance system 1. The user interface 6 may include at least one display performing simultaneously as data input means, e.g., a touch screen. Via the user interface 6, the assisted person may indicate his confidence manually in his ability to address the current scenario in the dynamic environment as an alternative to the exclusively automatic assistance method. Alternatively, the assisted person may explicitly request assistance by the assistance system 1.

The processor 4, in particular, performs the steps of the assistance method as discussed with reference to FIGS. 1 and 2.

The processor 4 provides the modulated audio output signal to the infotainment system 7. Alternatively, the processor provides the modulated audio output signal to an output driver 8.

The output driver 8 outputs the modulated audio output signal to a plurality of loudspeakers 9.1, 9.2, which output the modulated audio output signal to the assisted person as a perceivable sound from a spatially discernible location of origin or direction.

The infotainment system 7 may include the plurality of loudspeakers 9.1, 9.2 not explicitly shown in FIG. 2 spatially arranged in an interior of the ego-vehicle 1, in particular in a passenger cabin of the ego-vehicle 1. Using loudspeakers of the infotainment system 7 requires a set of loudspeakers, which enable to provide acoustic stimulus signals to the assisted person, which the assisted person can distinguish with regard to the spatial origin, thus the direction from which the acoustic stimulus arrives at the location of the assisted person. The invention may therefore enhance the functionalities of a vehicle infotainment system to fulfill or complement functionalities of an assistance system.

Alternatively or additionally, the processor 4 provides the modulated output signal via the output driver to a headphone assembly, which the assisted person may wear.

The headphone assembly may be part of a smartphone or a portable music player in specific embodiment. In this embodiment, the loudspeakers 9.1, 9.2 are part of the headphone assembly. The infotainment system 7 and the output driver 8 may be part of the smartphone or portable music player.

The processor 4 modulates the audio output signal by adapting a perceivable origin of the reproduced output sound such that the apparent origin moves gradually towards a direction or to a position defined in the direction of the determined spatial signal. In particular, the processor 4 modulates the obtained audio output signal with the spatial signal, wherein a perceived position of origin or a direction of the modulated audio output signal corresponds to the position information or the direction information included in the spatial signal.

The sensors 2.1, 2.2, 2.3 may include microphones arranged on the outside of on the ego-vehicle 10 in order to capture environmental sound from exterior environment, and in particular sound originating from the direction of the predicted behavior of the object or predicted event, thus originating from the spatial direction of the risk from the ego-vehicle's 10 point of view. The processor 4 may control playback of the captured environment sound using loudspeakers 9.1, 9.2 of the infotainment system 7 inside the ego-vehicle 10. In particular, the processor 4 may control playback of the captured environment sound as a modulated sound signal that corresponds to the modulated output signal with an adjusted relative sound volumes for individual loudspeakers 9.1, 9.2 of the infotainment system 7 inside the ego-vehicle 10. Thus, the processor 4 generates a virtual spatial sound origin perceived by the assisted person that corresponds to the direction or position information defined in the spatial signal.

Additionally, the processor 4 may control the at least one modulated audio output signal depending on a predicted level of urgency, with a varying precision of the sound as perceived by the assisted person. Varying a precision of the sound as perceived by the assisted person may ensure that the apparent location of the output sound aligns more specifically with the direction of the predicted risk. The spatial signal may modulate the audio output signal such that the larger the risk is measured or classified, the output sound is concentrated to a smaller angular region in direction of the predicted risk. Alternatively, in case of a smaller measured or classified risk, the output sound is concentrated to a wider angular region in direction of the predicted risk. By making this precision in directionality of the output sound depending on the value on the relevance measure of the determined risk, instead of, for example, being always at the highest possible level, a negative complacency or an attention tunneling effect on the assisted person is avoided. Such negative complacency or an attention tunneling effect would result in limiting the assisted person's attention early to a narrow region already for low values of the relevance measure or risk levels.

Additionally or alternatively, the processor 4 may control the modulation of the audio output signal such that an output volume level of the at least one output modulated audio output signal increases with an increasing deviation measure or until reaching a predetermined maximum value. The increase may be continuous or stepwise. A stepwise increase could be preferable to achieve an increased contrast between steps. Discretization could also be utilized selectively as an attention capturing mechanism in case a user fails to notice/react an issue appropriately.

The processor 4 of the assistance system 1 may include an ambient sound augmentation function that is adapted to generate artificial sound. Artificial sound may be stored as artificial sound files in the memory 5. The generated artificial sound files may be semantically associated with predetermined behaviors of objects or events in the environment. Generating and storing artificial sound files enables to overcome the problem that other traffic participants may not necessarily produce sound that the sensors 2.1, 2.2, 2.3 are able to capture, especially over large distances in a potentially noisy traffic environment.

The sensors 2.4, 2.5 acquiring sensor data from the interior of the ego-vehicle 1 may comprise active and/or passive sensors, e.g., acoustic sensors 2.4 (microphones). This enables to implement in the processor 4 processing, which not only makes the spatial signal depending on the determined values deviation, but optionally also on information about the assisted person. In particular, the assistance system 1 may determine the assisted person's state of attention, a vigilance of the assisted person and an attention history of the assisted person and generate the spatial signal further based on at least one of the determined assisted person's state of attention, vigilance of the assisted person and attention history of the assisted person.

The camera sensor 2.5 may provide image data, which enables monitoring head- and/or eye movement of the assisted person. The processor 4 may be configured to estimate which aspects of a current scene in the environment the assisted person perceives. The processor 4 may determine gaze patterns and fixation durations of the assisted person based on the image data provided by the camera sensor 2.5 to refine probability estimates for the perception of different elements in the environment, and in particular on the predicted behavior of objects and predicted events involving these objects.

The sensors 2.4, 2.5 acquiring sensor data from the interior of the ego-vehicle 1 may comprise sensors for determining other physiological measures, such as a pupil-dilation, a heart rate and a skin conductance, which enable to estimate the vigilance of the assisted person.

The processor 4 may use the determined information on the assisted person's attention and the vigilance of the assisted person to tailor the provided assistance to the assisted person to its estimated actual needs, according to a determined scene context for the environment and stored data about human behavioral patterns. The assistance system 1 may amplify a stimulus modulation by the modulation signal further whenever the assistance system 1 determines the vigilance of the assisted person, or the determined attention of the assisted person to the task is lower than a threshold that corresponds to a predetermined minimum attention or vigilance level required.

The determined attention level or the vigilance of the assisted person may further be used by the assistance system 1 for adapting a gradient of the modulation signal after the relevance measure or the risk level significantly decreases. For example, if a determined vigilance of the assisted person reaches regular levels well ahead of the reduction of the modulation signal after a risk has been resolved, the gradient of the modulation signal, and thereby the slope of the recovery, may be increased.

Alternatively, the assistance system 1 may determine the assisted person's individual need for support to require maintaining high modulation levels of the spatial signal unless the assisted person shows a sufficiently high level of vigilance. This processing results in enforcing a safety margin in the assistance to the assisted person.

The assistance system 1 may include a user interface 6. The user interface 6 may enable a manual input by the assisted person, for example by using a button press, by rotating a switch, by inputting characters via touch-sensitive area or a speech command with the user interface 6.

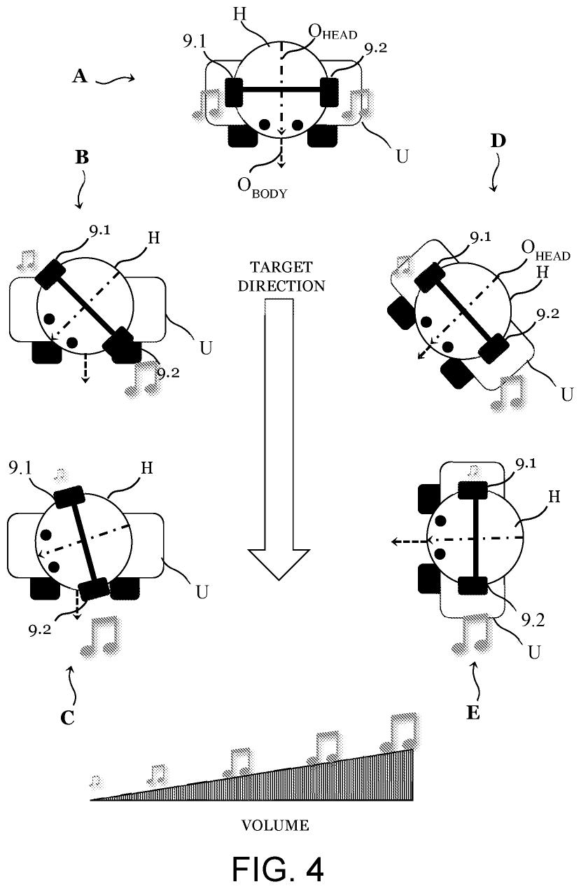

FIG. 4 illustrates an embodiment with an encoding adapted to limited hardware. Hardware limitations may concern speaker setups, earphones or headphones that lack the capability of reproducing sound such that the listener or wearer may distinguish between a perceived origin that varies between a front to rear directions, although distinguishing between varying directions between a left direction and a right direction for the perceived origin of the reproduced sound is possible. Portion A of FIG. 4 displays the example of a user U, corresponding to an assisted person, which wears a headphone device with loudspeakers 9.1 (right speaker) and 9.2 (left speaker) on its head H, which enables reproduction of stereo sound. The assistance system 1 is configured to drive the loudspeakers 9.1, 9.2, each of the right speaker and the left speaker independently from each other.

The assistance system 1 further comprises a rotation sensor as part of the sensors 2.1, 0.2.2, 2.3. The rotation sensor may include, e.g., a geomagnetic orientation sensor, a gyroscope, an accelerometer, or even a visual-based rotation sensing system. If not available, orientation sensors integrated with smartphones or other mobile computing devices may provide sensor signals that include orientation information in their sensor signal. Arranging the rotation sensor attached to the head H of the person enables to obtain orientation information on the spatial orientation of the head H of the assisted person U. In case the rotation sensor is arranged attached to a body of the person H enables to obtain orientation information on the spatial orientation of the body or a body part wearing the rotation sensor. In case of the rotation sensor of the smartphone, the assisted person may be required to rotate the smartphone in order to perform direction sampling.

When a head orientation O_{HEAD} is parallel to a target direction, as in portion A of FIG. 4, a front corresponding to the face of the assisted person U points in direction of the target direction, and the back of the head H is arranged opposite to the target direction. In portion A, the output sound signal by the loudspeakers 9.1, 9.2 includes no spatial modulation. Instead, only a sound modulation for an illusion of distance may be generated using changes in sound volume and filtering of the sound using auditory filters, e.g., low-pass filtering, adding reverberation on both channels.

FIG. 4 illustrates a strength of the spatial modulation of the sound of the channels driving loudspeakers 9.1, 9.2 by the respective size of the note symbols associated with the respective loudspeaker 9.1, 9.2. The larger a size of the note symbol, the larger the strength of the modulation of the corresponding channel driving loudspeakers 9.1, 9.2. An increased strength of modulation of a first channel relative to a second channel corresponds to an increase in shift of the perceived origin of the output sound towards the first channel.

When the assisted person U rotates the head H relative to the target direction as shown in portions B and C of FIG. 4, a spatial bias of the output sound towards of the loudspeaker 9.1, 9.2 closest to the target direction is added. Portions B and D of FIG. 4 illustrate this by the respective size of the note symbols respectively associated with the loudspeakers 9.1, 9.2. The spatial modulation reaches its maximum level when the head orientation U_{HEAD} is orthogonal to the target direction. The proposed encoding of the direction has the advantage that the assisted person U enjoys a balanced sound while moving into the correct direction, the target direction, and can quickly sample direction information by performing a short head-rotation in case it desires assistance concerning its current heading.

Portions D and E of FIG. 4 illustrate the corresponding relationships between an orientation O_{BODY} of a body of the assisted person U, the orientation O_{BODY} of the head O_{HEAD} of the assisted person U and the target direction. In portions D and E, the head orientation O_{HEAD} and the body orientation O_{BODY} are parallel to each other.

A maximum rotation of the head H needed to identify the target direction with a stereo setup for driving the audio output having two channels, each channel driving a respective loudspeaker 9.1, 9.2 independently from the other channel, is 90°.

A mono-setup for driving the audio output by the loudspeakers 9.1, 9.2 has only one channel driving both loudspeakers 9.1, 9.2 with a same level of the modulation strength of the acoustic signal.

The maximum required rotation of the head H needed to identify the target direction is 1800 for the mono-setup. When moving towards the opposite of the target location, a distance modulation, e.g., modulating the volume of the sound and filtering, increases, while moving in the target direction towards the target location decreases the modulation strength.

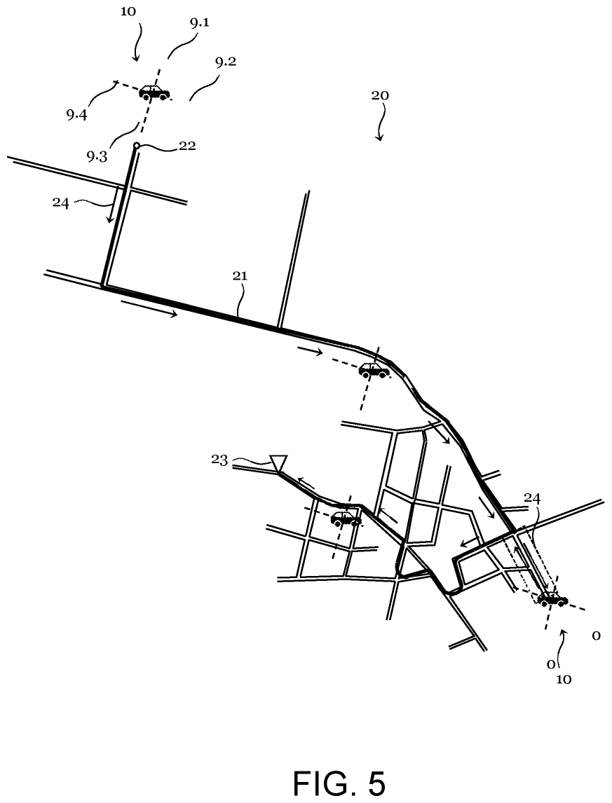

FIG. 5 presents a first exemplary scenario of applying the assistance method in a traffic environment. The first scenario illustrates applying the spatial output sound modulation of the assistance system for navigation purposes in an ego-vehicle 10. The ego-vehicle 10 includes an infotainment system, which reproduces an audio output signal and outputs the reproduced an audio output signal via four loudspeakers 9.1, 9.2, 9.3, and 9.4 arranged in the interior of the ego-vehicle 10.

Utilizing the method in a pedestrian navigation application is possible in an entirely analogous manner as discussed with reference to vehicle-based application illustrated in the scenario of FIG. 5.

In the scenario of FIG. 5, the perceived spatial location of the output sound is modified to support the assisted person in staying on a proposed route 21 from a starting location 22 to a target location 23 (destination). As long as the ego-vehicle 10 moves along the proposed route 21 shown on a road map 20, the assistance system 1 determines a deviation of zero between the acquired current location of the assisted person and the target location on the proposed route 21. Alternatively, the assistance system 1 determines a deviation of zero, or the deviation being smaller than a predetermined threshold between the estimated future location of the assisted person and the estimated future target location on the proposed route 21. The directional information included in the generating a spatial signal reflects this deviation of zero and results in a modulated audio output signal reproduced in the interior of the ego-vehicle 10, which corresponds to an unmodulated audio output signal as illustrated in FIG. 5 by the four speaker symbols being present around the representation of the ego-vehicle 10.

In the scenario of FIG. 5, the perceived spatial location of the output sound is modulated to support the assisted person operating the ego-vehicle 10 in returning to the proposed route 21, when a deviation between the current location of the assisted person and the ego-vehicle 10 and the spatial location of the proposed route 21 is determined. Alternatively, the assistance system 1 determines a deviation different from zero, or exceeding the predetermined threshold between the estimated future location of the assisted person and the estimated future target location on the proposed route 21. In the scenario of FIG. 5, the method determines such deviation in the region 24. In the region 24, the ego-vehicle 10 misses a turn and proceeds driving straight on the current road until stopping, reversing and moving back to the proposed route 21. As the ego-vehicle 10 moves along the proposed route 21 shown on the road map 20, the assistance system 1 determines a deviation between the acquired current location of the assisted person and the target location on the proposed route 21 that is different to zero or larger than the predetermined threshold. The directional information included in the generating a spatial signal reflects this deviation and results in a modulated audio output signal reproduced in the interior of the ego-vehicle 10, wherein a perceived position of origin or a direction of the modulated audio output signal corresponds to the position information or the direction information included in the spatial signal. In the region 24 marked in FIG. 5, only two of the four speaker symbols spatially arranged in a direction from the representation of the ego-vehicle 10 towards the proposed route 21 are shown. This indicates that the assisted person inside the ego-vehicle 10 is presented with audio output information from the direction of the proposed route 21 predominantly or even exclusively. Thus, as soon as leaving the proposed route 21, the assisted person is presented with an acoustically perceivable information due to the shift in perceived location of origin of the reproduced audio output sound, and moreover, receives a subtle hint on the direction of the proposed route 21 as seen from the current location of the assisted person.

In an entirely corresponding manner of the scenario of FIG. 5, the assistance system 1 may modulate the perceived spatial location of the output sound to support the assisted person operating the ego-vehicle 10 in selecting a predetermined direction, e.g., at intersections.

In an equally corresponding manner, the assistance system 1 may modulate the perceived spatial location of the output output sound to support the assisted person operating the ego-vehicle 10 in identifying an alternative proposed route 21 to the destination 23.

In further corresponding manner, the assistance system 1 may modulate the perceived spatial location of the output sound to support the assisted person operating the ego-vehicle 10 in identifying a destination 23 or a point of interest or points of interest.

The discussed embodiments utilizing a spatial sound modulation of audio output sound allows the respective information of interest to be conveyed in a subtle and intuitive manner without interfering with visual perception of the assisted person.

As long as the ego-vehicle 10 is on the proposed route 21 or location, the perceived sound appears to be centered on the assisted person. The assistance system 1 may modulate the audio output signal when arriving at or shortly before arriving at intersections, at or before suggested lane crossings, or at or before other locations that require a direction change, in order to generate the impression for the assisted person that the perceived sound appears to shift towards a preferred direction.

In case of leaving the proposed route 21, the perceived origin of the sound appears to lag behind on the proposed route 21 or to follow the ego-vehicle 10 at some distance to indicate that the proposed route 21 has been left until the ego-vehicle 10. Outputting the modulated audio output signal with the modified virtual location of sound origin ceases when the ego-vehicle 10 returns to the proposed route 21. Alternatively outputting the modulated audio output signal with the modified virtual location of sound origin may cease when a new proposed route 21 has been identified by a navigation system. In that case, a new modulated audio output signal may shift the perceived virtual sound location of the output audio output signal to indicate the new proposed route 21 to the assisted person.

When the assisted person reaches a destination 23 or a point of interest, but passes by the destination 23 or a point of interest, the perceived origin of the sound may be amended to appear to stay at the destination or point of interest. Additionally or alternatively, the perceived origin of the sound may be amended to move towards the destination or point of interest over a predetermined time.

It is immediately apparent that the assistance system 1 is applicable in various forms of mobility, including pedestrians, cyclists, or motorcyclists exceeding the road traffic scenario and operating a vehicle shown in the example of FIG. 5.

For example, the required location information location data may be acquired via a wearable device such as a smartphone, a smartwatch, a GPS tracker or similar sensor-equipped devices. The location information may be acquired from external sensors attached to devices in the environment, including other traffic participants.

Wearable speakers, in particular headphones, or external speakers that allow for perceived sound location modulation may be utilized for outputting the modulated audio output sound. In case of using speakers 9.1, 9.2 with low spatial resolution of the perceived location of origin of output sound, e.g., mono sound, alternative encodings of the position information or the direction information included in the spatial signal may be utilized. Alternative encodings may include modulating a volume of the audio output signal or applying specific auditory filters on the audio output signal. The alternative encodings may suffer from a potential cost of losing intuition for the assisted person, however come at the advantage of broadening possible implementations of the disclosure to low cost sound reproduction system.

Navigation applications or other objectives that rely on an indication of spatial points or directions of interest and that involve other forms of mobility may benefit from the assistance system. Alternative implementations offer assistance for various mobile activities, such as skiing, scuba diving, marine navigation, aerospace navigation, while the assisted person is using audio sound sources that enable corresponding implementations of the assistance system 1 and benefit from the output of a modulated audio signal.

In an example, a scuba diver may be guided along a preferred path and be pulled by a spatially modulated audio signal in case an undesired drift, resulting from a current or from buoyancy for ascent or descent, occurs.

Yet another example includes a skier that the assistance system 1 pulls to a predetermined part of a slope. The predetermined part of the slope may be less dangerous due to exposure avalanche. The assistance system 1 may pull the skier to an alternate part of the slope in order to avoid a colliding trajectory with another skier.

In a further advantageous embodiment, the assistance system 1 may support an athlete in weight/direction shifts, pace, acceleration, and deceleration while exercising by a respectively modulated audio output signal output via headphones worn by the athlete during exercise.

The assistance system 1 may support the athlete by including in the target state information at least one of time targets and velocity targets. The assistance system 1 generates the directional information including the directional information such that the output modulated output signal shifts the perceived location of origin of the modulated output signal in relation to the athlete towards a moving direction of the athlete. This aims at prompting the athlete to accelerate, e.g., to increase his velocity or to reduce a backlog in relation to an allocated target time. Alternatively, the assistance system 1 generates the directional information including the directional information such that the output modulated output signal shifts the perceived location of origin of the modulated output signal in relation to the athlete contrary to the moving direction of the athlete. This aims at prompting the athlete to decelerate, e.g., to reduce his velocity in order to avoid early exhaustion based on a relation of the current to an associated target time for a location on a running profile.

Supporting an athlete as discussed is widely similar to applying the system for fitness goals and for health-related goals.

Some embodiments utilize spatial audio output signal modulation to assist the assisted person in reaching temporal targets instead of or in addition to spatial navigation targets. For instance, while the assisted person is running or performing respective movement-related exercises, the output modulated audio output signal may be modulated such that the location of origin of the output sound appears to be centered to the location of the assisted person whenever the assisted person moves at a predetermined target speed. The output modulated audio output signal may be modulated such that the location of origin of the output sound appears to move towards a location that is ahead in a moving direction of the assisted person in case the assisted person moves slower than the predetermined target speed. When the assisted person returns to moving at the predetermined target speed, the assistance system 1 may also return to outputting the modulated audio output signal modulated such that the location of the origin of the output sound appears to be centered to the location of the assisted person. The effect of the assistance system 1 acting in this manner is that the assisted person receives an implicit motivation to catch up with the reproduced audio output signal.

In an entirely corresponding manner, in case of the assisted person moving faster than defined by a velocity target, the assistance system 1 may pull back the assisted person by shifting the perceived location of origin of the output audio output sound to rear of the assisted person, meaning to a location contrary to the moving direction.

In a specific embodiment of the assistance system 1, an option for the assisted person overriding a target set in the target state information via the user interface 6, e.g., via an application program (app), may exist. Alternatively, the assisted person ignoring persistently may be interpreted as overriding the target. Overriding a target may mean that the assistance system 1 ceases in generating the directional signal or reduces a strength of the directional signal indicating a deviation between the acquired location information and the acquired target location information, or between the acquired state information and the acquired target state information. Thus, the output modulated audio output signal is again centered on the current location of the assisted person or, alternatively, the strength of the location information is reduced.

The assistance system 1 integrated within fitness related applications is described with respect to encouraging the assisted person to maintain a target velocity. The assistance system may couple the discussed modulation of the audio output signal with further state targets for reaching objectives including navigation targets and safety targets. In particular, combining target information to include further at least one of navigation targets and targets derived from other knowledge sources, e.g., maps disclosing terrain-related information, route profile, and personal information on the assisted person, predetermined velocity profiles may vary during a training session. The assistance system may prioritize health-related goals, e.g., maintaining a predetermined heart rate or heart range and determine temporal targets based on the health-related goals for the state information.

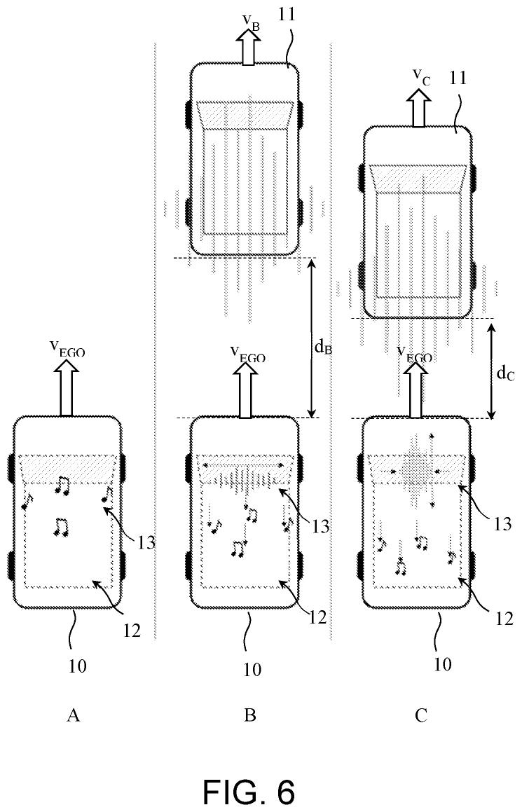

Navigation-based modulations of the audio sound signal may be combined with modulations of the audio sound signal for achieving other objectives as illustrated in FIG. 6. Other objectives discussed with reference to FIG. 6 may include safety-related aspects in two simple traffic scenarios.

The left portion of FIG. 6 displays the ego-vehicle 10 moving with an ego-velocity v_{ego} along a lane of a road. The ego-vehicle includes an infotainment system 7, and arranges a plurality of loudspeakers 9.1, 9.2, 9.3, 9.4 not explicitly shown in FIG. 6 for outputting audio output sound 13 into the interior of the ego-vehicle 10.

A specific example B in shown in the center portion of FIG. 6 may include shifting the perceived origin of the output output sound 13 towards the rear of the ego-vehicle 10 when a detected safety distance d_{B} to another vehicle 11 driving in front of the ego-vehicle 10 on a same lane drops below a minimum value.

A further example C shown in the right portion of FIG. 6 includes shifting the perceived origin of the output output sound towards 13 the rear of the ego-vehicle 10 when another vehicle 11 driving in front of the ego-vehicle 10 on a same lane starts braking, resulting in a rapidly decreasing distance dc between the ego-vehicle 10 and the other vehicle 11.

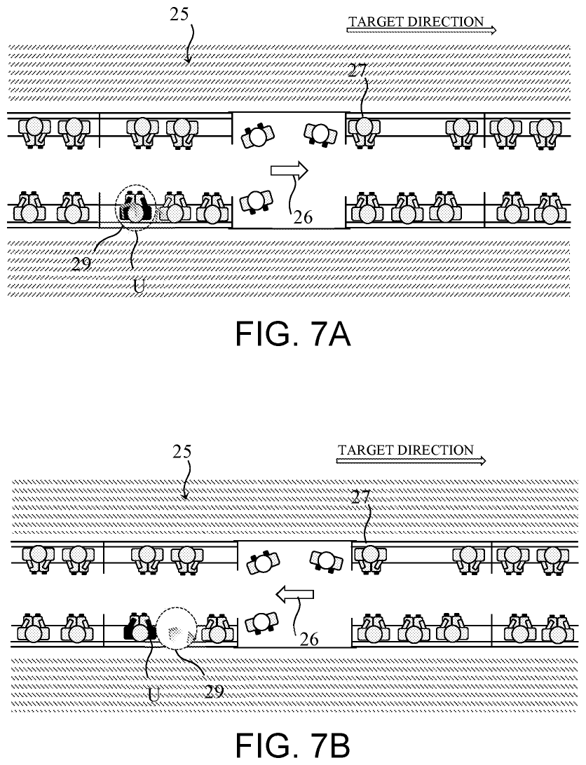

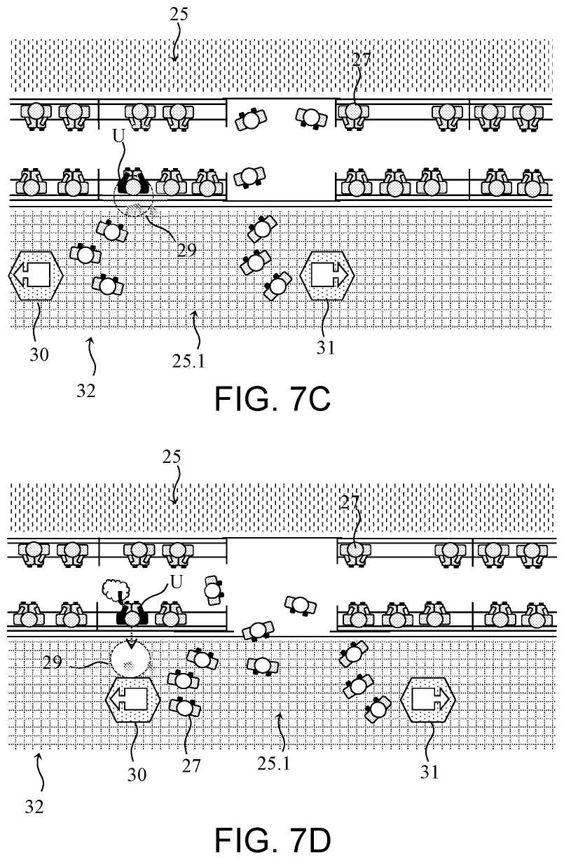

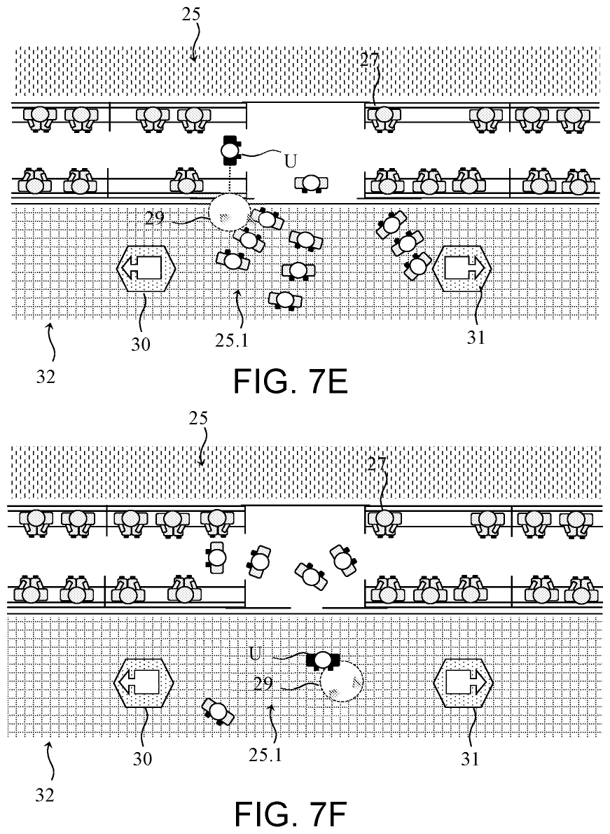

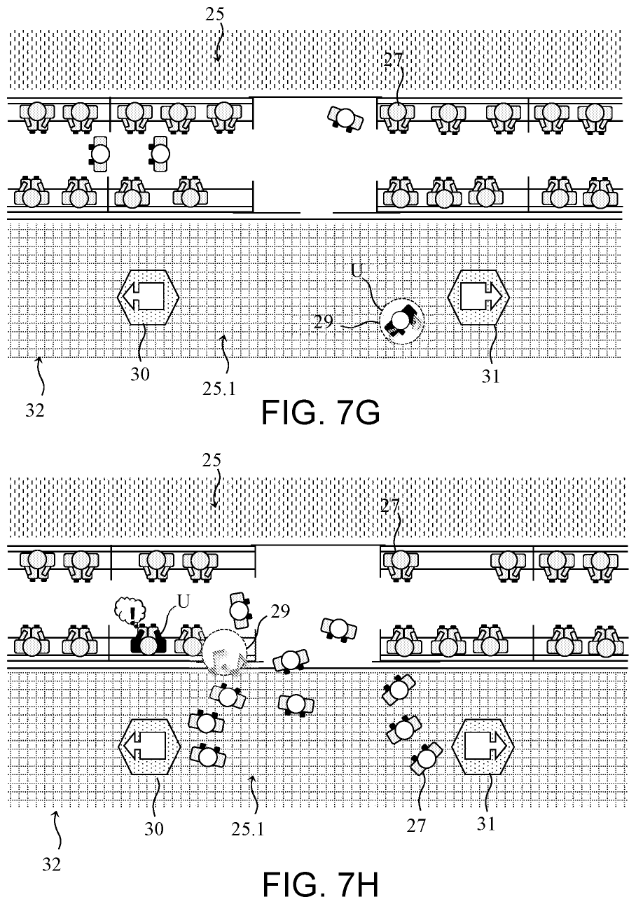

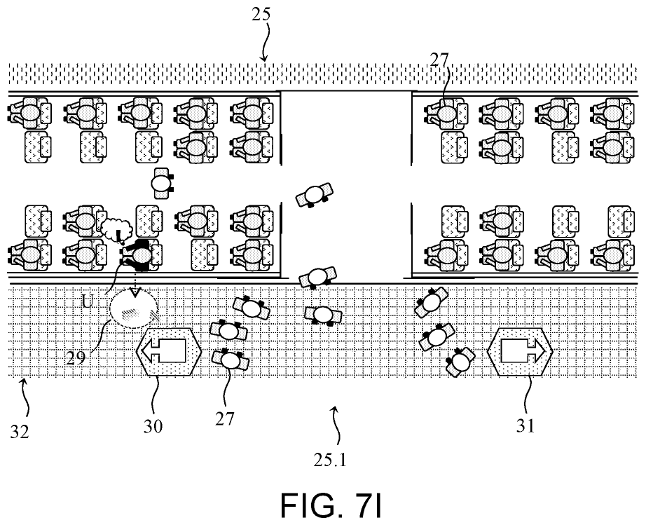

FIGS. 7A to 71 present exemplary scenarios of applying the assistance method in a public traffic environment. The scenario includes a carriage 25 of a train or a metro line moving into a direction 26 along the train tracks. FIGS. 7A to 71 show the carriage 25 in a top view with passengers 27 in some figures sitting or standing in the carriage 27, situated on a platform 32, and entering or exiting the wagon 25 via the carriage doors 25.1.

FIG. 7A provides a top view of the interior of the carriage 25 of a public transport system while travel into the direction 26. The public transport system may be rail-bound, e.g., a train, a tram, a metro, or road-bound, such as a bus. An assisted person U is sitting in the carriage 25, assisted by an embodiment of the method, e.g., running on mobile computing device, e.g., a smartphone, and listening to reproduced sound via a set of loudspeakers 9.1, 9.2.

In the scenario of FIG. 7A, the assistance system 1 determines the movement direction 26 or route to correspond to the target direction. The mobile computing device may determine the movement direction based on sensor signals from its sensors 2.1, 2.2, 2.3. The assistance system 1 determines the target direction, or the correct train line based on navigation instructions input by the assisted person U, for example. Alternatively, the assistance system 1 may determine the target direction or the correct train line based on an assumed navigation of the assisted person based on a stored navigation history, or acquired schedule data.

As the determined movement direction 26 and the target direction do not deviate, the assistance system 1 controls the audio output 29 via the loudspeakers 9.1, 9.2 to convey a perceived origin of the sound being centered onto the position of the assisted person U.

FIG. 7B shows the top view of the interior of the carriage 25 of a public transport system while travel into the direction 26. Nevertheless, the actual movement direction 26 of the train including the carriage 25 has changed into the opposite direction when compared with FIG. 7A. The scenario of FIG. 7B alleges that the target direction of the assisted person U has not changed with respect to the scenario depicted in FIG. 7A.

The mobile computing device may determine the movement direction based on sensor signals from its sensors 2.1, 2.2, 2.3 as in the scenario in FIG. 7A. Likewise, the assistance system 1 determines the target direction, or the correct train line based on navigation instructions input by the assisted person U, for example. The assistance system 1 determines the determined movement direction 26 and the target direction to differ, thus the assistance system 1 controls the audio output 29 via the loudspeakers 9.1, 9.2 to convey a perceived origin of the sound being shifted in the direction of the target direction and opposite to the movement direction from the position of the assisted person U. Hence, the assisted person U is alerted to the fact that a wrong train may have been entered, or the assisted person U missed a reversal in the movement direction 26 of the train.

FIG. 7C shows the top view of the interior of the carriage 25 while arriving at a target station. The target station has the platform 32 to the lower side of the carriage 25 in the scenario of FIG. 7C. The carriage 25 is still moving and the carriage doors 25.1 are not yet unlocked and closed.

The assistance system 1 determines arrival at the target station based on sensor signals from its sensors 2.1, 2.2, 2.3., e.g., a location sensor and a movement sensor. The assistance system 1 determines the target station based on navigation instructions input by the assisted person U, for example. Alternatively, the assistance system 1 may determine the target station or the correct train line based on an assumed navigation of the assisted person based on a stored navigation history, or acquired schedule data.

The HMI implemented via the modulated sound output controls the sound-balance between the loudspeakers 9.1, 9.2 to shift slightly towards the direction of the platform 5 from the center of the assisted person U in order to indicate that the train approaches the target station. Alternatively or additionally, the assistance system 1 may modulate the output sound to appear slightly more distant by an additional filtering via audio filters than in the scenario of FIG. 7A.

FIG. 7D shows the further evolvement of the scenario of FIG. 7C. The train has entered target station, has come to a stop, the carriage doors 25.1 have unlocked and opened. However, the assisted person U displays no measurable reaction to the arrival at the target station and, in particular, the assistance system 1 acquires no measurable reaction to the previously output auditory cue of FIG. 7C. Hence, the assisted person may miss the opportunity to exit the train at the target station.

In the scenario of FIG. 7D, the assistance system 1 may, in a specific example of user modeling, note both a failure to react appropriately to the needs of the situation and to react to the communicated auditory cues issued by the assistance system 1. Based on the failures of the assisted person U, the assistance system 1 controls in FIG. 7D the HMI to shift the sound-balance more strongly towards the direction of the platform 25 from the location of the assisted person U than in the scenario of FIG. 7C. Thus, the assistance system 1 achieves via the HMI the effect of communicating to the assisted person U a more pressing need to exit the carriage 25 noticeable.

Alternatively or additionally, the assistance system 1 may modulate the output sound to appear significantly more distant by an additional filtering via audio filters than in the scenario of FIG. 7A, and even more distant than in the scenario of FIG. 7C when just entering the station.

Optionally, the assistance system 1 may control an output balance shift combined with a supplementary auditory stimulus, e.g., outputting explicit navigation instructions, outputting an amplified sound recorded from the exterior, or reproducing sound files streamed by resp. recorded and communicated from devices present in the train or at the station, e.g., including announcements.

FIG. 7E shows a further evolvement of the scenario of FIGS. 7C and 7D at the station. Finally, the assisted person U starts to move and is on its way towards the carriage doors 25.1 and onto the platform 32.

The assistance system 1 controls the HMI to output sound with a balance still biased towards the direction of the platform 32 and slightly distanced to the center of mass of the assisted person U as long as the assisted person U is still on the train.

FIG. 7F shows the further evolvement of the scenario of FIG. 7E at the station. The assisted person U exited the train in time before the carriage doors 25.1 closed and is currently on the platform 32. According to navigation instructions derived from the planned route, the assisted person U needs to go towards destination 31 next, e.g., to another platform, where another train may start in order to continue the journey along the planned route.

However, the assisted person might not yet know in which direction to move on, and first needs to orient himself. This lack of orientation exhibits itself in hesitation of the assisted person U. The assistance system 1 senses the hesitation based on sensor signals, e.g., by a sudden stop to move, or short rotations of the head H or the body. The assistance system 1 provides the information obtained by the sensors 2.1, 2.2, 2.3 to a stored user model. The user model determines, whether the assisted person U knows where to move next.

The user model may use thresholds for determining based on the obtained sensor data, whether the assisted person shows signs of hesitation or other behaviors, and for concluding that the assisted person may benefit from assistance. The thresholds for determining whether the assisted person may benefit from or even requests assistance may depend on application context, scenario context, and on the particular assisted person U.

In case the assistance system 1 determines that the assisted person U may benefit from assistance, the system controls the HMI accordingly. In FIG. 7f, in the particular application context of the mixed mobility application of using public transport for the journey, and the scenario context of the assisted person U is determined to show signs of hesitation, the system 1 control the HMI output accordingly: the sound balance of the sound output by the loudspeakers 9.1, 9.2 shifts its perceived location of origin towards the target direction corresponding to the destination 31 in relation to orientation O_{HEAD} of the assisted person U. Hence, the assistance system 1 enables the assisted person U to get information for the correct direction for continuing his or her journey without having to check a phone display of a smartphone or to search for and interpret the signs at the station.

FIG. 7G shows the further evolvement of the scenario of FIG. 7F at the station. The assisted person U exited the train and proceeded on the platform 32 according to navigation instructions towards destination 31. In particular, the assisted person U followed the navigation hint provided by the output sound location and the direction information conveyed thereby, and is now on track to the destination 31 that corresponds to the next target location.

The assistance system 1 determines a movement of the assisted person U towards the correct direction, and concludes based thereon, an awareness of the assisted person U about where to head for the near future.

The assistance system 1 controls the output sound to have balance that is re-aligned with the assisted person U whose path the assistance system 1 predicts not to deviate from the target path in the near future. The re-aligned sound output balance corresponds to the perceived origin of the sound being centered on the assisted person U.

The re-aligned sound output implicitly provides the assisted person with a feedback on its advance on the target path, in particular on being on track towards his or her final (final) destination 23.

FIG. 7H shows an evolvement of the scenario of FIG. 7D, presenting an alternative to the scenario depicted in FIG. 7E. The train had entered the station, as discussed with reference to FIG. 7C, and the carriage doors 5.1 had opened but the assisted person U has not yet reacted to the arrival at the station and the previous auditory cue, as discussed with reference to FIG. 7E, and now the possibility to miss the opportunity to exit arises.

The assistance system 1, in response to determining arrival at the station, and determining that the assisted person U does not react to the previous auditory cue, controls the audio output 29 to shift the sound-balance more strongly towards the direction of the carriage door 25 to communicate the need to exit the carriage 25 swiftly to the assisted person U. Additionally, in the example of FIG. 7H, the assistance system 1 controls the modulated output sound to appear more distant from the location of the assisted person U by applying audio filters.

Alternatively or additionally, the assistance system 1 may control the HMI to output sound, in particular infotainment sound, with a shifted balance combined with outputting a supplementary auditory stimulus, e.g., including explicit navigation instructions to the assisted person U, outputting amplified recorded sounds from the exterior of the train carriage 25, for example, or reproducing sounds streamed, possibly recorded by, devices in the train or at the station, which may, e.g., include, e.g., public announcements.

FIG. 7I shows a scenario of the train at the station with open carriage doors 25.1 towards the platform 32 for entering or leaving the carriage 25. As in the scenario of FIG. 7H, the assisted person U has not yet reacted to the arrival at the station, and the previous auditory cue and may now miss the opportunity to exit the carriage 25 at the target station.

The assistance system 1, implemented in a mobile computing device, e.g., a smartphone, estimates seating direction information while the train moves. Alternatively or additionally, the assistance system 1 requests from an external server seating direction information and receives the requested seating direction information in response. The assistance system 1 controls spatial modulation, e.g., a leftward bias in relation to a face direction of the assisted person U in FIG. 7I according to a determined seating direction of the assisted person U.

The assistance system 1 of FIG. 7I may use a stereo audio setup in combination with acquired navigation information.

Alternatively or additionally, the assistance system 1 controls the output sound to include infotainment with a balance shift as shown in FIG. 7I, further supplemented by an added auditory stimulus, e.g., including at least one of an explicit navigation instructions, an amplification of outside sounds, or playing of sounds streamed by/recorded from devices in the train/station (e.g., announcements).

All features described above or features shown in the figures can be combined with each other in any advantageous manner within the scope of the disclosure and as covered by the appended claims, which define the invention. The detailed discussion of embodiments presents numerous specific details for providing a thorough understanding of the invention, which the attached claims define. It is evident that putting the claimed invention into practice is possible without including all of the specific details.

In the specification and the claims, the expression “at least one of A and B” may replace the expression “A and/or B” and vice versa due to being used with the same meaning. The expression “A and/or B” means “A, or B, or A and B”.

What is claimed is:

1. A computer-implemented method for assisting a person to perform a task, the method comprising steps of:
acquiring location information on a location of the assisted person or state information on a state of the assisted person;
acquiring target location information on a target location based on a spatial target of the assisted person or target state information on a target state of the assisted person;
generating a spatial signal based on the acquired location information and the acquired target location information, or based on the acquired state information and the acquired target state information, wherein the spatial signal includes a position information or a direction information;
obtaining an audio output signal for reproduction to the assisted person;
modulating the obtained audio output signal with the spatial signal, wherein a perceived position of origin or a direction of the modulated audio output signal corresponds to the position information or the direction information included in the spatial signal; and
outputting the modulated audio output signal to the assisted person.

2. The computer-implemented method according to claim 1, wherein
in the step of generating the spatial signal, the spatial signal is generated based on a deviation of the acquired location information from the acquired target location information, or based on a deviation of the acquired state information and the acquired target state information.

3. The computer-implemented method according to claim 1, wherein
the position information corresponds to the current location of the assisted person in case of determining that the deviation of the acquired location information from the acquired target location information, or the deviation of the acquired state information and the acquired target state information is below a first predetermined threshold or zero.

4. The computer-implemented method according to claim 1, wherein
generating the spatial signal includes setting the position information to correspond to a location ahead of the location of the assisted person in a moving direction of the assisted person.

5. The computer-implemented method according to claim 1, wherein
generating the spatial signal includes setting the position information to correspond to a location in a direction of the target location from the location of the assisted person in case of determining that a moving velocity of the assisted person decreases or that the moving velocity of the assisted person is below a second predetermined threshold or the assisted person stops.

6. The computer-implemented method according to claim 2, wherein
the state of the assisted person includes an ego-velocity of the assisted person, and the target state includes a predetermined velocity target, or
the state of the assisted person includes a safety-related variable associated with the assisted person, and the target state includes a predetermined target value of the safety-related variable, or
the state of the assisted person includes a fitness-related variable associated with the assisted person, and the target state includes a predetermined target value of the fitness-related variable, or
the state of the assisted person includes a health-related variable associated with the assisted person, and the target state includes a predetermined target value of the health-related variable.

7. The computer-implemented method according to claim 6, wherein
the fitness-related variable or the health-related variable includes a heart rate of the assisted person, and the predetermined target value of the fitness-related variable or the predetermined target value of the health-related variable includes a predetermined heart rate target, and
generating the spatial signal based on the heart rate and the acquired target heart rate, wherein the spatial signal includes setting the position information or the direction information based on the deviation of the heart rate and the acquired target heart rate.

8. The computer-implemented method according to claim 7, wherein
generating the spatial signal based on the heart rate and the acquired target heart rate, determining the position information or the direction information based on the deviation of the heart rate and the acquired target heart rate, and further based on a pace target value.

9. The computer-implemented method according to claim 6, wherein
the assisted person moves in a dynamic traffic environment including a plurality of other participants
the safety-related variable includes a distance of the assisted person to the other traffic participants, and the predetermined target value of the safety-related variable includes a minimum distance of the assisted person to the other traffic participants.

10. The computer-implemented method according to claim 6, wherein
the assisted person wears a virtual reality headset for moving in a virtual representation and reproducing the audio output signal associated with the virtual representation, and
the safety-related variable includes a location of the assisted person in a real environment of the assisted person, and the predetermined target value of the safety-related variable includes a safe location of the assisted person.

11. The computer-implemented method according to claim 1, wherein
outputting the modulated output signal via plural speakers or earphones worn by the assisted person.

12. The computer-implemented method according to claim 1, wherein the method comprises a step of
unified encoding of multiple objectives in the target state information, wherein individual contributions of the objectives are weighted differentially based on at least one of priority constraints at least one of preset user preferences.

13. The computer-implemented method according to claim 12, wherein
the target state information encodes objectives that include at least one of safety objectives, navigation objectives, sports objectives, health objectives, and convenience objectives.

14. The computer-implemented method according to claim 12, wherein the priority constraints include a legally required preference for safety objectives over convenience objectives.

15. The computer-implemented method according to claim 1, wherein the method comprises steps of
determining a reaction or a lack of reaction of the assisted person to a previously output modulated audio output signal modulated based on a same spatial signal; and
adapting a parameter of the modulated audio output signal based on the determined reaction or lack of reaction of the assisted person.

16. The computer-implemented method according to claim 1, wherein the method comprises,
in the step of acquiring location information, acquiring orientation information of an orientation of the assisted person in relation to at least one of the locations of the assisted person and a target direction of the assisted person, and
in the step of generating the spatial direction signal, generating the spatial direction signal further based on the acquired orientation information.

17. A computer program product comprising machine-readable instructions executable by a digital processor to cause the digital processor to control a method for assisting a person to perform a task, the method comprising steps of:
acquiring location information on a location of the assisted person or state information on a state of the assisted person;
acquiring target information on a target location based on a spatial target of the assisted person or target state information on a target state of the assisted person;
generating a spatial signal based on the acquired location information and the acquired target location information, or based on the acquired state information and the acquired target state information, wherein the spatial signal includes a position information or a direction information;
obtaining an audio output signal for reproduction to the assisted person;
modulating the obtained audio output signal with the spatial signal, wherein a perceived position of origin or a direction of the modulated audio output signal corresponds to the position information or the direction information included in the spatial signal; and
controlling output of the modulated audio output signal to the assisted person.

18. A device for assisting a person to perform a task, the device comprising:
at least one sensor configured to acquire location information on a location of the assisted person or state information on a state of the assisted person;
at least one interface configured to acquire target location information on a target location based on a spatial target of the assisted person or target state information on a target state of the assisted person;
at least one processor configured to generate a spatial signal based on the acquired location information and the acquired target location information, or based on the acquired state information and the acquired target state information, wherein the spatial signal includes a position information or a direction information,
to obtain an audio output signal for reproduction to the assisted person,
to modulate the obtained audio output signal with the spatial signal, wherein a perceived position of origin or a direction of the modulated audio output signal corresponds to the position information or the direction information included in the spatial signal, and
to control outputting the modulated audio output signal to the assisted person via plural speakers or via earphones worn by the assisted person.
